(19) United States
(12) Patent Application Publication
(10) Pub. No.: US 2026/0344717 A1
(43) Pub. Date: Nov. 5, 2026

(54) CELL-CULTURED EDIBLE FISH PRODUCTS

(71) Applicant: BlueNalu, Inc., San Diego, CA (US)

(72) Inventors: Lauran Rebecca MADDEN, San Diego, CA (US); Chandrashekhar SONAR, San Diego, CA (US)

(73) Assignee: BlueNalu, Inc., San Diego, CA (US)

(21) Appl. No.: 19/115,461

(22) PCT Filed: Sep. 23, 2023
(86) PCT No.: PCT/US2023/074971

§ 371 Date: Mar. 26, 2025

Related U.S. Application Data
(60) Provisional application No. 63/377,059, filed on Sep. 26, 2022.

(51) Int. Cl. A23L 17/00

(52) U.S. Cl. CPC A23L 17/00

(57) ABSTRACT

Provided herein are in vitro cultured fish compositions, methods and systems to culture cells derived from a fish source.

### BACKGROUND

Cell-cultured food products are part of food alternatives which have been the focus of development by numerous companies around the world as a means to address public health, environmental and animal welfare issues associated with animal farming and agriculture. Chemically contaminated and spoiled foods have serious implications on the health of individuals. Foodborne illness and food poisoning have different origins—bacteria, virus, parasites, mold, contaminants, etc. Indeed some cases of food poisoning can be traced back to chemical and natural toxins. One of the toxins targeted by the Food and Drug Administration (FDA) and European Food Safety Authority (EFSA) is the biogenic amine histamine. Biogenic amines (BAs) in food constitute a potential public health concern due to their physiological and toxicological effects. The consumption of foods containing high concentrations of biogenic amines has been associated with health hazards. The harmful effects range from minor gastric problems to major health fatalities. Chemical contaminants are strongly linked with severe consequences, lack of personal control, and long-term effects (Kher et al., 2011). According to the WHO, more than 200 diseases are transmitted by food and the vast majority of the population will contract a foodborne disease at some point in their lifetime. For example, in the U.S. 48 million people (one in six) suffer a foodborne disease each year. Of these, 128,000 are hospitalized and 3000 die from such diseases. (Claudia Ruiz-Capillas et al., 2019). Food consumption is the most likely source of human exposure to metals. Metals such as cadmium and lead can easily enter the food chain. Heavy metals can seriously deplete specific nutrients in the body that can decline the immunological defenses, impair psycho-social facilities, and cause intrauterine growth retardation. Heavy metal consumption is also associated with malnutrition and increases the rates of gastrointestinal diseases (Khan et al., 2008). Food contaminants are also a leading cause of cancer (Abnet, 2007). Polychlorinated biphenyls (PCBs) exposure due to food contamination can adversely affect children's neurological development and the immune response (Schantz et al., 2004). Pesticides in the food as contaminants also show severe health implications. Excessive levels of these chemicals in food cause neural and kidney damage, congenital disabilities, reproductive problems, and can prove to be carcinogenic (Bassil et al., 2007). The accumulation of pesticides in the tissues of the body can also result in metabolic degradation (Androutsopoulos et al., 2013). There is also the risk of neurodevelopmental disorders like attention deficit disorders, autism, cerebral palsy and mental retardation caused by industrial chemicals like arsenic, PCBs, and lead in both food and water. The present application provides edible fish products that are substantially free of contaminants and with extended shelf life compared to conventional products.

### SUMMARY

The present invention relates to cell-cultured food products derived from a fish source and related cells. Compositions, are also provided. In at least one embodiment, the fish source is Bluefin tuna, and the edible product formed therefrom is substantially free of contaminants

The edible composition provided herein may contain less than 0.1 parts per million [ppm] of contaminant. The contaminants may be environmental contaminants (e.g., mercury), food processing contaminants, unapproved adulterants and food additives. Environmental contaminants include mercury, lead, cadmium, zine, copper, nickel, chromium, arsenic, aluminum, fluoride, radon, and pesticides.

According to an aspect of the invention, provided herein are edible compositions that are substantially free of contaminants and comprise a homogenous mixture of in vitro cultured fish cells. The cells may each be less than from about 20 μm to about 200 μm in diameter. In one embodiment, the cells are each less than 20 m in diameter. In another embodiment the cells are each less than 200 μm in diameter.

The edible composition provided herein comprises one, two or three cell types. The cell types may be myoblasts, myotubes, fibroblasts, endothelial cells, neurons, red blood cells, pre-adipocytes, induced pluripotent stem cells, adipocytes, or a combination thereof.

The edible composition may be in the form of a viscous slurry, in accordance with an aspect of the present invention. The edible composition may be frozen or freeze-dried.

The edible composition may be a liquid, semi-liquid, semi-solid, solid, or foam.

According to another aspect, the edible composition comprises a collection of non-fibrous in vitro cultured fish cells, wherein the cells are in a layered formation, and each layer contains a homogenous mixture of cells are described. The edible composition may comprise a single cell type or may multiple cell types (e.g., 1-3 cell types, 4 or more cell types). In another embodiment, the edible composition comprises a collection of non-fibrous in-vitro cultured tuna cells arranged in a homogenous formation with no layering. In yet another embodiment the non-fibrous in-vitro cultured cells are Bluefin tuna cells.

According to another aspect, a method is described, for making a substantially contaminant-free Bluefin tuna slurry. The method comprises harvesting myoblasts and fibroblasts from muscle tissue of a wild caught Pacific Bluefin tuna, harvesting preadipocytes from subcutaneous fat of a wild caught Pacific Bluefin tuna, expanding the myoblasts, fibroblasts, and preadipocytes in vitro for at least 50 population doublings to achieve a stable cell line, expanding the myoblasts and/or preadipocytes under suitable growth conditions, transitioning the myoblasts into differentiation media to form myocytes or myotubes, treating the preadipocytes with lipid-containing media to form adipocytes differentiating the myoblasts and/or preadipocytes in dilute differentiation liquid, and concentrating the cells by centrifugation, settling, or other separation methods to separate the cells from the liquid, thereby forming a slurry comprising a cell concentration of at least 10⁶ cells per ml. The slurry may comprise about 10⁶ cells/ml to about 10⁹ cells/ml. The method may further comprise freezing or drying the slurry to form a solid product. The wild caught Pacific Bluefin tuna used in the methods described herein may weigh between about 12-200 pounds.

The slurry used in the methods described herein may comprise one or more cell types, for example, myoblasts, myotubes, pre-adipocytes, adipocytes, fibroblasts, endothelial cells, embryonic derived cells, or induced pluripotent stem cells.

The expanding step may comprise seeding 0.1-1 gram of tissue/well.

Treating the preadipocytes may cause the cells to transition from small, proliferating cells to rounded and non-proliferating, lipid-loaded cells. The growth conditions may include a pH range of 7.3 to 7.5, and temperature changes from 15 °C to 30 °C.

The culture media, and systems herein described and related compositions, cells, cell biomass, and cell-cultured food products, herein described may be used in connection with various applications wherein cell viability, controlled proliferation and contamination levels in cell and related cell-cultured food product is desired. For example, the compositions-herein described and related cells, cell biomass, and cell-cultured food products, herein described may be used to generate cell-cultured food products such as food products that are substantially free of contaminants. Accordingly, exemplary fields of applications comprise food manufacturing, food processing and commercialization. Additional exemplary applications include uses of the culture media, compositions, methods and systems and related cells and cell biomass cell-cultured food products herein described in several fields including basic biology research, applied biology, bioengineering, bioenergy, medical research, therapeutics, and in additional fields identifiable by a skilled person upon reading of the present invent

The details of one or more embodiments of the invention are set forth in the accompanying drawings and the description below. Other features, objects, and advantages will be apparent from the description and drawings, and from the claims.

### BRIEF DESCRIPTION OF THE DRAWINGS

FIGS. 1A-IG show a schematic representation of marbling seen in the cell-cultured Bluefin tuna products of the invention.

FIGS. 2A and 2B show the extended shelf-life of the cell-cultured Bluefin tuna product compared to conventional Bluefin tuna at 4 °C. The degree of color change is substantially slower in the cell-cultured product.

FIG. 3 shows the shelf-life stability of cell-cultured Bluefin tuna product at −80 °C. The graph shows the color difference in cell-cultured Bluefin tuna following storage at −80 °C for eleven days. CIELAB values, a* and b*, were measured for cell-cultured Bluefin tuna (BN BFT).

FIG. 3 shows no difference in color (a*/b*) between day 0 and day 11, indicating extended shelf-life stability of the cell-cultured Bluefin tuna product at −80 °C storage conditions.

FIG. 4 is a graph showing the color difference in cell-cultured Bluefin tuna compared to conventional Bluefin tuna following storage at −20 °C for three months. Color difference of conventional Bluefin tuna (Conventional BFT) and cell-cultured Bluefin tuna samples (BN BFT) was quantified as ΔE (the total color difference) calculated in the CIELAB color space system, which measures three values: the achromatic component L* (light vs. dark), and two color descriptors, the a* (red vs. green) and b* (yellow vs. blue) values. Statistical analysis was performed using two-way ANOVA. The graph shows a significant reduction in color change (p<0.05) between the cell-cultured Bluefin tuna compared to conventional Bluefin tuna indicating extended shelf-life of cell-cultured Bluefin tuna product under frozen storage conditions.

FIG. 5 shows the extent of lipid oxidation in the cell-cultured Bluefin tuna product compared to conventional Bluefin tuna following storage at 4 °C for two weeks. Oxidation was measured by assaying the amount of Malondialdehyde (MDA) per sample for conventional Bluefin tuna (Conventional BFT) and cell-cultured Bluefin tuna (BN BFT) at 4 °C for 14 days (n=3). The graph shows a significant decrease (p<0.05) in the amount of MDA production in cell-cultured Bluefin tuna compared to conventional Bluefin tuna by two-way ANOVA, indicating reduced oxidation in cell-cultured Bluefin tuna product under refrigeration storage conditions.

### DETAILED DESCRIPTION

Provided herein are cell-cultured edible food products derived from a fish source, compositions, methods and systems and related cells, and cell biomass. In one embodiment, the cells are derived from one or more primary cell lines isolated from wild-caught fish, for example, from wild-caught Bluefin tuna.

The term “about” when referring to a measurable value such as an amount, a temporal duration, and the like, is meant to encompass variations of ±20% or less, or in some instances ±15% or less, or in some instances ±10% or less, or in some instances ±5% or less, or in some instances ±1% or less, or in some instances ±0.1% or less, from the specified value, as such variations are appropriate.

The term “cell line” is a term of art that refers to a defined population of cells that can be maintained in culture for an extended period of time. A “stabilized cell line” is a cell line that exhibits genomic and phenotypic stability, doubling time below 96 hours, and viability of over 70% after 24 hours of seeding.

The term “myoblast” is a term of art that refers to precursors of myocytes, which are also called muscle cells. Myoblasts differentiate into muscle cells through myogenesis as will be understood by a person skilled in the art. Myoblasts can be classified as skeletal muscle myoblasts, smooth muscle myoblasts, and cardiac muscle myoblasts depending on the type of muscle cell that they will differentiate into. Exemplary myoblasts of aquatic animals comprise skeletal muscle myoblasts and smooth muscle myoblasts.

The term “fibroblast” is a term of art that refers to type of cell in the connective tissue of animals and that synthesize components of the extracellular matrix, such as collagen. Fibroblasts produce the structural framework for animal tissues and play a critical role in wound healing. Fibroblasts are the most common cells of connective tissue in animals. Fibroblasts have a branched cytoplasm surrounding an elliptical, speckled nucleus having two or more nucleoli. Active fibroblasts can be recognized by their abundant rough endoplasmic reticulum. Inactive fibroblasts, also called fibrocytes, are smaller, spindle-shaped, and have a reduced amount of rough endoplasmic reticulum. Although disjointed and scattered when they have to cover a large space, fibroblasts, when crowded, often locally align in parallel clusters. Exemplary fibroblasts include fibroblasts from muscle and other tissues such as brain, heart or skin.

The term “adipocyte” is a term of art that refers to fat cells, which are also known as lipocytes. Adipocytes are the cells that primarily compose adipose tissue, specialized in storing energy as fat. Adipocytes can be derived from mesenchymal stem cells which give rise to adipocytes through adipogenesis. In cell culture, adipocytes can also form osteoblasts, myocytes and other cell types. There are two types of adipose tissue, white adipose tissue (WAT) and brown adipose tissue (BAT), which are also known as white and brown fat, respectively, and comprise two types of fat cells. Adipocytes can arise either from preadipocytes resident in adipose tissue, or from bone-marrow derived progenitor cells that migrate to adipose tissue. Cells used herein typically comprise adipocytes from white adipose tissue.

The term “preadipocyte” is a term of art that indicates progenitors of mature differentiated adipocytes which can be stimulated to form adipocytes. Preadipocytes can be isolated from subcutaneous or visceral fatty tissue of an animal.

Preadipocytes can be grown in a preadipocyte growth medium which contains all the growth factors and supplements necessary for the optimal growth of undifferentiated preadipocytes. For example, the preadipocytes can be grown in a preadipocyte growth medium containing endothelial cell growth supplement, epidermal growth factor, hydrocortisone, and/or heparin.

The formation of adipocytes from preadipocytes involves a tightly regulated cell differentiation process, referred to as adipogenesis, in which mesenchymal stem cells commit to preadipocytes and preadipocytes differentiate into adipocytes. The terms “differentiate,” or “differentiation,” refer to a process of a change of expression patterns during which multipotent gene expression alters to cell type specific gene expression. Transcription factors, such as peroxisome proliferator-activated receptor γ (PPARγ) and CCAAT enhancer-binding proteins (C/EBPs) are main regulators of adipogenesis. Characteristic features of differentiated adipocytes include but are not limited to growth arrest, morphological change, high expression of lipogenic genes and production of adipokines such as adiponectin, leptin, resistin (in mice, not in humans) and TNF-alpha, as will be understood by a person skilled in the art.

As used herein, the term “homogeneous products” are products formed as single piece and that are substantially uniform throughout the piece with respect to their sensory and functional attributes.

As used herein, the term “media” refers to a composition in a liquid, solid or gel state comprising organic, inorganic and/or biogenic ingredients in which a cell is capable of surviving, maintaining vitality or proliferating. A medium typically comprises a basal medium.

The term “basal medium” as used herein indicates culture media comprising components essential for cell survival and growth such as amino acids, glucose, and ions such as calcium, magnesium, potassium, sodium, and phosphate, as will be understood by a person skilled in the art.

An example of basal medium is Basal Media Formulation (www.sigmaaldrich.com/life-science/cell-culture/learning-center/media-formulations/basal.html). Additional examples can be identifiable by a skilled person.

An exemplary biogenic ingredient includes serum. A media can be chemically defined. For example, Lipid Mixture 1, available from Sigma Aldrich (www.sigmaaldrich.com/catalog/product/sigma/10288?lang=en&region=US) contains non-animal derived fatty acids (2 μg/ml arachidonic and 10 μg/ml each linoleic, linolenic, myristic, oleic, palmitic and stearic), 0.22 mg/ml cholesterol from New Zealand sheep's wool, 2.2 mg/ml Tween-80, 70 g/ml tocopherol acetate and 100 mg/ml Pluronic F-68 solubilized in cell culture water.

A media in the sense of the invention can have biogenic ingredients including Fetal Bovine Serum (FBS) or cod liver oil fatty acids. For example, Lipid Mixture (1000×), available from Sigma Aldrich (www.sigmaaldrich.com/catalog/product/sigma/15146?lang=en&region=US) contains cholesterol, 4.5 g/L, cod liver oil fatty acids (methyl esters), 10 g/L, polyoxyethylenesorbitan monooleate, 25 g/L, and D-α-tocopherol acetate, 2.0 g/L.

Food “texture” is defined by the International Standards Organization (ISO) in their standard vocabulary for sensory analysis as “all the rheological and structure (geometrical and surface) attributes of a food product perceptible by means of mechanical, tactile, and where appropriate, visual, and auditory receptors' (ISO, 2008). Texture is a key quality parameter used in the fresh and processed food industry to assess consumer acceptability. Among the texture characteristics, hardness (firmness) is one of the most important parameters and is often used to determine the freshness of food. Springiness, cohesiveness, adhesiveness, and chewiness are also significant properties for the texture evaluation for meat-based products.

Food “color” is a physical attribute which is commonly associated with the quality of the food. The surface food color can be easily measured with instrument to be used as a quantitative quality tool. Over the life of the product color may change indicating degradation of the product quality. To analyze color changes in products the International Commission of Illumination (CIE) proposed a universal method in 1931 to be used in analyzing color. This method distinguishes color into three different tristimulus values. More recently the Munsell system simplifies quantifying color even further through a multidimensional method. L*, a*, b*, h, and C readings can be quantified and compared. L represents the overall lightness of a sample. a* value denotes redness or greenness in a sample, while b* value denotes yellowness and blueness. Hue (h) and Chroma (C) values are derived from a* and b* values with hue expressed in radians or degrees of the angle within the color space and chroma as a measure of intensity as distance from the achromatic center of the color space. Color analysis can indicate surface degradation of product and color difference value (ΔE) can be helpful in distinguishing the difference between storage treatments.

The Institute of Food Science and Technology (IFST) in the United Kingdom defined “shelf life” as “the period of time during which the food product will remain safe; be certain to retain desired sensory, chemical, physical, microbiological and functional characteristics; and comply with any label declaration of nutritional data when stored under the recommended conditions” (Shelf Life of Foods: Guidelines for Its Determination and Prediction, 1993).

“Substantially free” of heavy metal contaminants (e.g., mercury) means that the edible composition comprises less than 0.1 parts per million [ppm] of the one or more heavy metal contaminants (e.g., less than 0.1 ppm, less than 0.01 ppm, less than 0.001 ppm, or less than 0.0001 ppm).

### Cultured Fish Cells and Compositions

In certain embodiments of the present invention, culture media, methods and systems to culture a cell of a fish, for example, a cell of Bluefin tuna, are described, as well fish cells such as Bluefin tuna cells obtainable and/or obtained thereby. In embodiments of the present invention, compositions, methods and systems are described as well as related fish cells, fish cell biomass and fish cell-cultured food products with a controllable cell lipid content and lipid uptake, and/or with an improved cell differentiation and/or cell viability in connection with set lipid content and lipid uptake. The fish cell-cultured food products may be in multiple forms, including, for example, a slurry, mince, homogenous single cell, homogenous multi-cell, non-fibrous layered single cell, or non-fibrous layered multi-cell product.

In one embodiment, the source of the fish cells is any fish cell from various species.

In another embodiment the fish source is any one or more of the seven species of tunas in the genus Thunnus. These include the northern Bluefin tuna (T. thynnus), albacore (T. alalunga), yellowfin tuna (T. albacares), southern Bluefin tuna (T. thynnus maccoyii), bigeye tuna (T. obesus), blackfin tuna (T. atlanticus), and longtail tuna (T. tonggol). See https://www.britannica.com/animal/tuna-fish, which is incorporated by reference herein.

In another embodiment, the fish cells are sourced from the genus thynnus orientalis, wherein said fish cells are sourced from Bluefin tuna.

The cell-cultured fish products of the present invention differ from conventional fish derived products in several ways.

As opposed to conventional fish-derived products wherein the cells are oriented such that the muscle fibers and blood vessels are aligned, the cell cultured fish products disclosed herein comprise cells that are not oriented in an organized way. The cell-cultured layered products disclosed herein are similarly unorganized within each layer. The connectivity of the cells within the cell-cultured Bluefin tuna product is limited in terms of the number of cell connections and a low amount of extracellular matrix. In contrast, in a traditional fish derived product there are many cells connected to one another and an extracellular matrix. The amino acid profile and fatty acid profile of the cell-cultured fish may be controlled and modified. Moreover, the cell-cultured products may contain sugars, fibers, texturizing agents, natural colors, natural flavoring agents not found in conventional products. The color of the cell-cultured products may also be controlled by the addition of natural colorant compounds to cell culture media (e.g., carotenoids such as astaxanthin), or to the final product (e.g., anthocyanins, beet juice, beta-carotene, curcumin, spirulina, insect-derived colorants, including but not limited to carmine, carotenoids such as astaxanthin, heme, leghemoglobin, lycopene, monascus red, paprika or other natural colorants) to achieve the desired color (e.g., red, pink, white).

In certain embodiments, the invention relates to edible compositions that comprise in vitro cultured fish cells arranged in a homogenous or layered formation. The in vitro cultured fish cells are generally small in comparison to conventional fish cells. The cultured cells may each be less than 20 μm in diameter when proliferating, e.g., less than 20, 19, 18, 17, 16, 15, 14, 13, 12, 11, 10, 9, 8, 7, 6, or 5 μm in diameter. Differentiated cultured cells tend to be medium sized, as compared to a single myocyte with one nuclei that may be small (i.e., 10-30 μm) or much larger in vitro muscle tubes (i.e., 50-200 μm).

In certain embodiments, the cultured cells can be less than from 20 μm to 200 μm in diameter, such as less than 20 μm, less than 21 μm, less than 22 μm, less than 23 μm, less than 24 μm, less than 25 μm, less than 26 μm, less than 27 μm, less than 28 μm, less than 29 μm, less than 30 m, less than 31 μm, less than 32 μm, less than 33 μm, less than 34 μm, less than 35 μm, less than 36 μm, less than 37 μm, less than 38 μm, less than 39 μm, less than 40 μm, less than 41 μm, less than 42 μm, less than 43 μm, less than 44 μm, less than 45 μm, less than 46 μm, less than 47 μm, less than 48 μm, less than 49 μm, less than 50 μm, less than 51 μm, less than 52 μm, less than 53 μm, less than 54 μm, less than 55 μm, less than 56 μm, less than 57 μm, less than 58 μm, less than 59 μm, less than 60 μm, less than 61 μm, less than 62 μm, less than 63 μm, less than 64 μm, less than 65 μm, less than 66 μm, less than 67 μm, less than 68 μm, less than 69 μm, less than 70 μm, less than 71 μm, less than 72 μm, less than 73 μm, less than 74 μm, less than 75 μm, less than 76 μm, less than 77 μm, less than 78 μm, less than 79 μm, less than 80 μm, less than 81 μm, less than 82 μm, less than 83 μm, less than 84 μm, less than 85 μm, less than 86 μm, less than 87 μm, less than 88 μm, less than 89 μm, less than 90 μm, less than 91 μm, less than 92 μm, less than 93 μm, less than 94 μm, less than 95 μm, less than 96 μm, less than 97 μm, less than 98 μm, less than 99 μm, less than 100 μm, less than 101 μm, less than 102 μm, less than 103 μm, less than 104 μm, less than 105 μm, less than 106 μm, less than 107 μm, less than 108 μm, less than 109 μm, less than 110 μm, less than 111 μm, less than 112 μm, less than 113 μm, less than 114 μm, less than 115 μm, less than 116 μm, less than 117 μm, less than 118 μm, less than 119 μm, less than 120 μm, less than 121 μm, less than 122 μm, less than 123 μm, less than 124 μm, less than 125 μm, less than 126 μm, less than 127 μm, less than 128 μm, less than 129 μm, less than 130 μm, less than 131 μm, less than 132 μm, less than 133 μm, less than 134 μm, less than 135 μm, less than 136 μm, less than 137 μm, less than 138 μm, less than 139 μm, less than 140 μm, less than 141 μm, less than 142 μm, less than 143 μm, less than 144 μm, less than 145 μm, less than 146 μm, less than 147 μm, less than 148 μm, less than 149 μm, less than 150 μm, less than 151 μm, less than 152 μm, less than 153 μm, less than 154 μm, less than 155 μm, less than 156 μm, less than 157 μm, less than 158 μm, less than 159 μm, less than 160 μm, less than 161 μm, less than 162 μm, less than 163 μm, less than 164 μm, less than 165 μm, less than 166 μm, less than 167 μm, less than 168 μm, less than 169 μm, less than 170 μm, less than 171 μm, less than 172 μm, less than 173 μm, less than 174 μm, less than 175 μm, less than 176 μm, less than 177 μm, less than 178 μm, less than 179 μm, less than 180 μm, less than 181 μm, less than 182 μm, less than 183 μm, less than 184 μm, less than 185 μm, less than 186 μm, less than 187 μm, less than 188 μm, less than 189 μm, less than 190 μm, less than 191 μm, less than 192 μm, less than 193 μm, less than 194 μm, less than 195 μm, less than 196 μm, less than 197 μm, less than 198 μm, less than 199 μm, or less than 200 μm in diameter.

The fish cells are also substantially free of contaminants (e.g., environmental contaminants). Exemplary environmental contaminants include microplastics; persistent organic pollutants (POPs) such as polychlorinated biphenyls (PCBs), organochlorines (OCs) and polybrominated biphenyl ethers (PBDEs); heavy metals such as mercury, lead, cadmium, zine, copper, nickel, chromium, arsenic, aluminum; fluoride; radon; microplastics; parasites, bacteria, and pesticides.

In some embodiments, the edible composition is substantially free of mercury and arsenic. Substantially free of heavy metal contaminants (e.g., mercury) means that the edible composition comprises less than 0.1 parts per million [ppm] of the one or more heavy metal contaminants (e.g., less than 0.1 ppm, less than 0.01 ppm, less than 0.001 ppm, or less than 0.0001 ppm).

In addition to tuna cells derived from the genus Thunnus, particularly Bluefin tuna cells, cells from a wide variety of fish species can be used. Such species include but are not limited to bass, flounder, hake, scup, smelt, rainbow trout, hardshell clam, blue crab, peekytoe crab, spanner crab, cuttlefish, Eastern oyster, Pacific oyster, anchovy, herring, lingcod, moi, orange roughy, Atlantic Ocean perch, Lake Victoria perch, yellow perch, European oyster, Dover sole, sturgeon, tilefish, wahoo, yellowtail, sea urchin, Atlantic mackerel, sardines, black sea bass, European sea bass, hybrid striped bass, bream, cod, drum, haddock, hoki, Alaska pollock, rockfish, pink salmon, snapper, tilapia, turbot, walleye, lake whitefish, wolffish, hardshell clam, surf clam, cockle, Jonah crab, snow crab, crayfish, bay scallop, Chinese white shrimp, sablefish, Atlantic salmon, coho salmon, skate, dungeness crab, king crab, blue mussel, greenshell mussel, pink shrimp, Escolar, chinook salmon, chum salmon, American shad, Arctic char, carp, catfish, dory, grouper, halibut, monkfish, pompano, abalone, conch, stone crab, American lobster, spiny lobster, octopus, black tiger shrimp, freshwater shrimp, gulf shrimp, Pacific white shrimp, squid, barramundi, cusk, dogfish, kingklip, mahi-mahi, opah, mako shark, swordfish, albacore tuna, yellowfin tuna, geoduck clam, squat lobster, sea scallop, rock shrimp, barracuda, Chilean sea bass, cobia, croaker, eel, blue marlin, mullet, sockeye salmon, Bluefin tuna, shrimp, crabs, lobster, and echinoderms (e.g. sea cucumbers and sea urchins).

Preferred aquatic animals include yellowtail (e.g., Seriola lalandi), mahi-mahi (Coryphaena hippurus), red snapper (Lutjanus campechanus), Bluefin tuna (e.g., Thunnus orientalis and Thunnus thynnus), yellowfin tuna (Thunnus albacares), cod (e.g., Gadus morhua, Gadus Macrocephalus, Gadus ogac), flounder, halibut, herring, mackeral, pompano, salmon, sea bass, Patagonian toothfish (Dissostichus eleginoides), squid, clams, lobster, crabs, scallops, shrimp, eel, bass (e.g., Micropterus salmoides), bluegill (Lepomis macrochirus), and carp (e.g., Hypophthalmichthys molitrix).

The homogenous or layered mixture of in vitro cultured fish cells disclosed herein contain only one, two, three or four different cell types, whereas conventional fish-derived products comprise many different types of cells, including myoblasts, myotubes, endothelial, neurons, red blood cells, adipocytes, and combinations thereof. The mixture of in vitro cultured fish cells may be formed into different product types, such as slurry, mince, homogenous or layered sheet, and homogenous or layered block. For example, in some embodiments a cell-cultured slurry or mince product contains one or two cell types. The different cell types included in the cell-cultured products include myoblasts, myotubes, fibroblasts, endothelial cells, neurons, red blood cells, pre-adipocytes, induced pluripotent stem cells, adipocytes, and combinations thereof.

The cell-cultured edible fish products provided herein may be frozen or refrigerated or freeze-dried, such that they may be used later.

The edible compositions may be liquid, semi-liquid, semi-solid, solid, or foam.

In various embodiments, cultured edible fish products according to the present invention may contain functional agents in addition to cultured fish cells, including one or more texture agents that provide firmness, mouthfeel and other textural characteristics like cohesiveness, springiness, and chewiness, and optionally additional functional agents that prevent peroxidation of lipid components, reduce microbial contamination and/or otherwise extend product shelf life, and optionally sensory agents such as flavorings and colorants; nutritional supplements.

### Functional Agents

### Texture Agents

Texture agents, and the concentration of each, can be chosen to achieve a degree of texture attributes mentioned above and characteristics suitable for the product's intended purpose. Product formulations may include one or more gelling agents and/or one or more thickening agents.

In addition to the above texture agents, product may contain protein isolates/concentrates/texturized proteins including but not limited to soy, wheat, pea, chickpea, hemp, water lentil, lentils, oat, rice, and potato. Also, it may include oils, fats, and/or hydrogenated vegetable oils and shortenings including but not limited to sunflower, safflower, rapeseed, canola, soybean, coconut, palm, and algal. These ingredients also contribute to the overall texture and mouthfeel of the product. In exemplary embodiments, the total concentration of agents contributing to texture can be between 1-40% (w/w) of final product weight.

Food-safe gelling agents suitable for use in the products include, but are not limited to, sodium alginate, carrageenans, agar, gellan gum, konjac gum, and curdlan gum, among others. Similarly, a variety of food-safe thickening agents suitable for use include, but are not limited to, guar gum, gum Arabic, locust bean gum, methyl cellulose, psyllium husk, pea fiber, citrus fiber, and xanthan gum, among others. The concentration of particular texture agents varies according to the intended use of the product and may range from 0.05% to 10%.

In addition to firmness, certain texture agents further provide additional functional attributes including, but not limited to, thermal stability and browning. Such agents allow the piece to remain solid during heating and/or brown during cooking to create an appetizing appearance and/or flavor. The concentrations of such agents are chosen to provide a product that is acceptably heat stable and/or that browns in a controlled manner, without being unpleasantly tough to consumers. In some embodiments texture agents are selected to provide emulsion stabilization, in addition to other sensory or functional attributes.

Some texture agents, such as locust bean gum and gellan, may have synergistic effects with respect to texture. Specific combinations, as well as the absolute and relative concentrations of the individual agents that comprise the combination, are selected to achieve a product that has a degree of firmness approximating conventional fish meat without sacrificing other desirable sensory or functional characteristics.

### Coloring Agents

In conventional Bluefin tuna, the sarcoplasmic protein, myoglobin, is responsible for the red color of the muscle. The amount of myoglobin and fat in the different cuts provide them with distinct colors ranging from dark red (akami) to pinkish red/pink (chutoro and otoro; commonly called as toro).

In the cell culture products, natural myoglobin is absent and therefore, natural coloring agents mentioned in the earlier embodiment can be used in the form of powder, liquid, or emulsion to achieve different shades of product as mentioned in above embodiment. The colorants can be used as such or in combinations to achieve desired result. In one example the concentration of color can vary from 0.001% to 5% to achieve L*, a*, and b* values in the range of 33.0-62.8, 4.77-21.9, and 4.72-15.4, respectively.

Only addition of natural colors does not ensure appropriate color, Bluefin tuna cells are responsible for adding some color characteristics as well as changing overall color tone. In one embodiment, the L*, a*, b* values of product changed from 50.18, 18.46, 14.30 to 69.89, 17.24, 14.26, respectively when added with Bluefin tuna cells. Similarly, Bluefin tuna cells texture may also be manipulated by changing the overall firmness of the edible product from 656 g to 442 g.

### Sensory Agents

In addition to texture agents, homogeneous and layered in vitro cultured fish cell products may optionally be formulated with one or more sensory agents, including, but not limited to, natural colorants, natural flavoring agents. Suitable colorants are those that provide the desired color at a pH generally found in meat obtained from living fish, i.e., a pH of from 6 to 7. Food-derived colorants are preferred for reasons of safety and consumer appeal, although colorants derived from insects or other sources that are safe for human consumption can also be used. Suitable colorants include, but are not limited to, food derived colorants such as anthocyanins, carotenoids, red radish powder, beet juice, leghemoglobin, lycopenes, monascus red and paprika, as well as insect-derived colorants, including but not limited to carmine.

The flavor of very fresh fish is characterized by mild, delicate aromas due in part to volatile 6-, 8-, and 9-carbon carbonyls and alcohols arising from the action of lipoxygenases on long-chain polyunsaturated fatty acids. In various exemplary embodiments, one or more of these compounds are added to further enhance the flavor provided by the cultured Bluefin tuna cells. The flavor agents may include vegetable oil, coconut oil, palm oil, algal oil, sunflower oil, safflower oil, soybean oil, olive oil, avocado oil, grapeseed oil, peanut oil, canola oil.

Flavor can also be modified through the use of salts including sodium chloride and potassium chloride; acidic ingredients such as vinegar, citric acid, tartaric acid, malic acid, folic acid, fumaric acid, and lactic acid, among others.

Texture agents provided as salts of sodium, potassium, calcium or magnesium can affect flavor aspects such as saltiness or bitterness. In such cases, the type and concentrations of other flavor agents, such as salts and alkaline ingredients, are adjusted so as to give the desired flavor profile.

In some cases, single cell Bluefin tuna products further include flavor agents that complement the flavor of fish, such as soy sauce, ginger, sesame, herbs such as parsley, dill, chervil and/or cilantro, among others; juice and/or extracts from citrus fruits such as calamansi, lemon, lime, orange, grapefruit, kumquat, and/or yuzu, among others; and/or black, red and/or white pepper, among others.

Flavorants can be added in their natural form, and/or as semi-dried or dry powders, encapsulations, extracts or absolutes, among others.

### Nutritional Supplements

Optionally, the nutritional content of the homogeneous in vitro cultured fish cell products provided herein may be supplemented, for example with amino acids, peptides, proteins and/or lipids, the latter including but not limited to, nutritional supplements, including amino acids, vitamins, minerals and/or carbohydrates can be provided in the cell culture media, which are then taken up and incorporated by the cells. Exemplary lipids include one or more of a polyunsaturated fatty acid, a saturated fatty acid and/or a sterol, alone or in combination with an effective amount of nervonic acid. In some embodiments, each supplemented lipid, e.g., polyunsaturated fatty acid, saturated fatty acid and/or the sterol, is present in a concentration of about 10 μg/ml or higher.

In other, alternative embodiments, nutritional supplements including lipids as described above, amino acids, vitamins, minerals and/or carbohydrates, as well as nutritional fiber, may be added to the cell culture media, or to the cell slurries after harvesting, or can be added during formulation of the product.

### Preservatives

Optionally, homogeneous in vitro cultured fish cell products can be formulated with agents that decrease microbial load and/or peroxidation, increase product shelf life and improve consumer safety. Examples of such agents include antioxidants suitable for use in producing homogeneous single cell type products, including but not limited to, 2,4,5-Trihydroxybutyrophenone (THBP), algal extracts, anoxomer, apigenin, ascorbic acid, baicalein, botanical extracts (including but not limited to blueberries, ginseng, goji berries, grape seed and green tea), butylated hydroxy toluene (BHT), butylated hydroxyanisole, butylated hydroxyanisole (BHA), carnosol, carotenoids, catalase, catechin, creatine, cilauryl thiodipropionate, epicatechin gallate, ethyoxyquin, lipoic acid, mitoquinol, morin, myricetin, Nacetylcysteine, phenolics, pinostrobin, proanthocyanidin dimer B2, propionyl-L-carnitine, propyl gallate, quercetin, resveratrol, rosemary extract, rutin, sauchinone, TBHQ (tertiary butylhydroquinone), tert butylhydroquinone (TBHQ), THBP (2,4,5-trihydroxybutyrophenone), thiazolidine, thiodipropionic acid, thymol and tocopherols.

In some embodiments, the antioxidants are plant-derived, including but not limited to algal extracts, apigenin, ascorbic acid, baicalein, botanical extracts (including, but not limited to, blueberries, ginseng, goji berries, grape seed and green tea, carnosol, carotenoids, catalase, catechin, epicatechin gallate, lipoic acid, morin, myricetin, pinostrobin, proanthocyanidin dimer B2, propionyl-L-carnitine, quercetin, resveratrol, rosemary extract, rutin, sauchinone, thymol and tocopherols.

### Contaminants

Bluefin tuna is an apex predatory fish and long-lived fish and accumulates mercury through dietary transfer. The tissue mercury concentrations in Bluefin tuna (BFT) often exceed threshold human consumption guidelines for large predatory fish of 1 μg·g−1 wet weight (w.w.) set by the US Food and Drug Administration and the World Health Organization. According to Tseng et. al. (2021), total mercury levels in the muscle tissue of Pacific Bluefin tuna (Pacific BFT) ranged from 0.49 to 5.65 μg·g−1 w.w. (mean=2.00±0.83 μg·g−1 w.w., n=261), ~94% of which exceeded the safe consumption guideline of 1 μg g−1 w.w. Also, Table 1 (n=19) summarizes the level of environmental contaminants including mercury and arsenic in the Pacific and Atlantic Bluefin Tuna samples. Refer to https://www.pnas.org/doi/10.1073/pnas.2111205118, which is incorporated in its entirety herein. Cell cultured Bluefin tuna according to the present invention will be significantly lower in all environmental contaminants. See Table 1 below showing naturally occurring levels in wild-caught and ranched bluefin tuna. “Significantly lower” levels refers to levels of any one or more environmental contaminants to be lower by at least 60% of the levels permitted by state and/or federal guidelines. Table 2 shows the level of heavy metals—arsenic, cadmium, lead, and mercury—in conventional (ranched) Pacific and Atlantic Bluefin tuna and cell-cultured Bluefin tuna (n=3).

TABLE 1
Metal content (n = 19) of wild-caught and ranched bluefin tuna
as detected by ICP-MS. All values shown in parts per billion (ppb).
Heavy metals mercury and lead were detected in all samples tested.

| Parts per billion | Chu-toro | O-toro |
|---|---|---|
| Arsenic (As) | 1490-4660 | 1960-9800 |
| Cadmium (Cd) | <10 | <10 |
| Mercury (Hg) | 195-633 | 179-423 |
| Lead (Pb) | <10 | <10 |
| Iron (Fe) | 3860-10200 | 3750-8860 |
| Manganese (Mn) | 143 | |
| Zinc (Zn) | 3410-5460 | 2740-4660 |
| Copper (Cu) | 150-323 | 123-291 |
| Tin (Sn) | <10 | <10 |
| Antimony (Sb) | <10 | <10 |
| Chromium (Cr) | 11.0-26.1 | 10.1-59.2 |
| Nickel (Ni) | 15.8-33.0 | 10.9-29.5 |
| Selenium (Se) | 259-571 | 203-618 |
| Molybdenum (Mo) | <10 | <10 |
| Silver (Ag) | <10 | <10 |
| Barium (Ba) | <10 | <10 |
| Beryllium (Be) | <10 | <10 |
| Cobalt (Co) | <10 | <10 |
| Thallium (Tl) | <10 | <10 |
| Vanadium (V) | <10 | <10 |
| Strontium (Sr) | 44.0-92.4 | 39.6-122.0 |

TABLE 2
Heavy metal contents (n = 3) of ranched Pacific and Atlantic
conventional Bluefin tuna and cell-cultured Bluefin tuna as
detected by ICP-MS. All values shown in parts per million (ppm).

| Parts per | Conventional | Cell-cultured |
|---|---|---|
| million, w/w | Bluefin Tuna | Bluefin Tuna |
| Arsenic (As) | 1.57 ± 0.26 | <0.01 |
| Cadmium (Cd) | 0.003 ± 0.00 | <0.001 |
| Mercury (Hg) | 0.412 ± 0.004 | <0.005 |
| Lead (Pb) | <0.01 | <0.01 |

### Storage Stability Studies

Stability of fish and fish products during refrigerated and frozen storage is affected by various conditions including packaging, temperature, relative humidity, temperature fluctuations, product composition, processing conditions etc. Major physical and chemical changes occur during refrigerated and frozen storage of fish and fish products like color degradation/change, lipid and protein oxidation/hydrolysis causing off flavors, production of potentially hazardous compounds such as histamine, textural changes like softening, nutrient losses, flavor and taste degradation, weight loss due to moisture dripping etc. Degradation of at least one attribute may be considered as a loss of shelf life.

In conventional Bluefin tuna color change (browning) and lipid oxidation are of concern limiting its refrigerated and frozen shelf life. Under prolonged low-temperature freezing storage, the color of tuna turns brown because of excessive metmyoglobin (MetMb) production, which negatively affects its commercial value and also results in food waste. Mb, which exists in muscle fiber cells, is the most important color substance in tuna (Singh, Benjakul, Zhou, Zhang, & Deng, 2021). In general, the bright red color of tuna muscle is mainly associated with the presence of red pigments, especially oxymyoglobin (OxyMb). However, OxyMb converts to the brown MetMb during processing and storage (Hoa et al., 2020). MetMb is mainly formed due to Mb oxidation, free radicals produced during lipid oxidation and trimethylamine oxide generation (Grunwald, Tatiyaborworntham, Faustman, & Richards, 2017). See also Ying Bu et al, Food Science and Technology (2022) and references cited therein. https://www.sciencedirect.com/science/article/pii/S0023643821018685

In one example, the ratio of a*/b* was significantly reduced in southern Bluefin tuna by 60% during first two days of refrigerated storage changing color from red to brown (Ying Bu et al. 2022). This study also suggests that the texture was softened with significant reduction in hardness (76%), chewiness (76%), springiness (11%), and resilience (45%) over the storage period of 6 days. The study also found significant increase in the volatile nitrogen compounds and lipid oxidation products during storage period suggesting unacceptable quality deterioration. In another example, a similar trend was observed for the color of Pacific Bluefin tuna stored in a refrigerator where the ratio of a*/b* was reduced by 88% over storage period of 10 days. Similar observations have been reported in Bluefin tuna stored under frozen conditions at −18 and −55 C. See https://www.ncbi.nlm.nih.gov/pmc/articles/PMC9368106/

In the case of cell culture Bluefin tuna (homogenous Bluefin tuna prototype) packed in parafilm and stored under refrigeration at 4 °C, the overall color degradation was at much slower rate with only 16% reduction in the a*/b* value over the refrigerated storage of 10 days.

In another example during frozen storage at −80 °C, the product packed in parafilm showed excellent freeze thaw stability with only <0.5% drip loss and retention of color attributes. It is noted that “weight loss” during frozen storage is important from an economic and quality perspective. Lower “weight loss” associated with the cell-cultured Bluefin tuna according to the invention is yet another benefit provided to the consumer. This is appearing to comport with the observations of Jinfeng Wang et al. (2022) wherein the data suggest that there was little quality change during short-term frozen storage at 18 °C. However, at a frozen storage temperature of −55 °C the bluefin tuna exhibited significantly improved quality compared with the frozen storage temperature of −18°. The contents of this reference are incorporated herein in its entirety.

In various embodiments, edible compositions according to the present invention may optionally include functional agents such as nutritional supplements, preservatives, pH modulators, emulsifiers, emulsion stabilizers, flowability enhancers, humectants and moisture retention agents, among others. Such agents provide a variety of benefits including but not limited to facilitation of manufacturing, product formation, moisture enhancement, moisture retention, and/or improved shelf life.

### Product Forms

Homogeneous and layered in vitro cultured fish cell products can be formed in a wide variety of shapes, including but not limited to saku block (i.e., uniform pieces suited for preparing sashimi and other sliced presentations, for pan searing or for further processing into mince, steaks, fillets and loins, sashimi, dice, slices and cubes.

### Slurry Product of In Vitro Cultured Fish Cells

In another embodiment of the invention, the in vitro cultured fish cell product is a slurry. The slurry may be a viscous slurry. Viscosity is a measure of a fluid's resistance to flow. A viscous slurry can be defined based on certain rheological studies to determine the critical parameters preferred in a cell-cultured slurry according to its intended purpose. For example, viscosity within a range between 25,000 cP to 50,000 cP. Viscosity outside of this range is also possible depending on the specific product application.

Slurries can be used in the manufacture of products that include, but are not limited to, nutritional supplements and food ingredients. In one embodiment, a nutritional supplement can be produced by filling capsules with freeze-dried cultured fish cells. Such capsules may be used in the same manner as fish oil capsules but offer the advantage of having a uniform and reproducible lipid profile while being free of environmental contaminants. The capsules themselves may be made from animal-free materials such as, for example, cell-cultured gelatin, modified tapioca starch and/or vegetable cellulose such as hydroxypropylmethylcellulose (also known as hypromellose), the latter of which offers the advantage of delaying release of their contents until they reach the small intestine, thereby optimizing lipid absorption.

In other embodiments, slurries can be used in fresh, frozen or dried form as food ingredients, alone or in combination with other animal and/or plant materials, for the production of a variety of foods, such as baby foods, pet foods and other products.

Slurries may be dispensed during manufacture of cultured edible product including for example Bluefin tuna cell products by a variety of methods, which are selected according to factors that may include but are not limited to viscoelastic properties, shear rate, pressure, temperature, and, in cases where mixing occurs simultaneously with dispensing, mixing requirements. Devices suitable for dispensing slurries include but are not limited to positive displacement pumps such as Rotary-type, internal gear, screw, shuttle block, flexible vane or sliding vane, circumferential piston, helical twisted roots (e.g. Wendelkolben pumps) or liquid ring vacuum pumps; piston pumps; auger pumps; peristaltic pumps; progressive cavity pumps; and extruders.

### Homogenous Single and Multiple Cell Type Products of In Vitro Cultured Fish Cells

Homogeneous single cell type products of the in vitro cultured fish cells of the invention can be produced using single cell type slurries that comprise cells selected from myoblasts, myotubes, fibroblasts, endothelial cells, neurons, red blood cells, pre-adipocytes, induced pluripotent stem cells and adipocytes, among others.

In yet another embodiment, homogenous multiple cell type products of in vitro cultured fish cells may be produced using multiple cell type slurries from cultures containing one, two, three, four or more cell types, such as myoblasts, myotubes, fibroblasts, endothelial cells, neurons, red blood cells, pre-adipocytes, induced pluripotent stem cells, adipocytes, and combinations thereof. Alternatively, multiple cell type products may be prepared from two, three, four or more single cell type slurries that are then combined in proportions that are suited to the intended purpose of the final product. In an exemplary embodiment according to the present invention, the cells are Bluefin tuna cells.

Multiple cell type products comprising both myocytes and adipocytes may be formulated to produce a product having a fat content that mimics the fat content of wild-caught or farmed fish cuts that are high in fat, medium-fat or low-fat (lean). In exemplary embodiments. Bluefin tuna cultured-cell products are formulated to mimic Bluefin tuna cuts characterized as otoro (high-fat), chutoro (medium-fat) or akami (lean). In some embodiments, multiple cell type products each having different proportions of myocytes and adipocytes may be combined to form a layered product that mimics the multi-layered natural Bluefin tuna cuts categorized as toro.

In certain embodiments, multiple cell type products provide a more desirable nutritional profile than products that contain only muscle. For example, multiple cell type products can be formulated using fish adipocytes that are lipid loaded, such that they provide a lipid profile that provides optimum nutritional benefits.

Homogenous single cell or multiple cell type products may be formed into blocks as shown in the schematic (g) that are further processed into mince, as described below. The products may also be formed as sheets of uniform or variable thickness that may then be combined into layered products, as described below.

Pieces served to consumers, such as center of plate fillets, sashimi and cubes (the latter including “poke” cubes served either as an appetizer or a main course) may be formulated with one or more texture agents, coloring agents, nutrient supplements and other food ingredients to mimic the texture, color, and other sensory and nutritional aspects of conventional Bluefin tuna flesh and that is pleasing to consumers.

Both single cell type and multiple cell type fish cultured-cell products intended for use as soft food diet products, such as infant and baby foods, and pet foods, may be formulated using one or more texture agents of a type, and in concentrations, chosen to create a semi-solid or semi-liquid product.

### Mince

Minces of in vitro cultured fish cells are optimally formed by processing large pieces formed from either single cell type or multiple cell type homogeneous products.

Single cell type or multiple cell type slurries may n be combined with one or more texturizers, or one or more binders, to produce a piece having a texture optimized for processing by chopping into small pieces, the size of which will vary according to the intended use of the mince. In an embodiment, the piece is partially or completely frozen as a preliminary step in order to facilitate chopping.

Because chopping will result in “breaking” the gel formed by the texture agents, consequently releasing moisture, pieces intended for use in producing mince may be formulated using small amounts of dry ingredients to facilitate water retention. Such dry ingredients include, but are not limited to, textured proteins, flours and gums, among others.

Two or more minces, each produced from pieces having different cell type compositions, can be combined according to the intended use of the final mince.

### Non-Fibrous, Layered Products of In Vitro Cultured Fish Cells

Embodiments of the present invention also include edible compositions that comprise a collection of non-fibrous in vitro cultured fish cells, wherein the cells are in a layered formation and substantially free of contaminants as shown in the schematic. The compositions comprise multiple layers (e.g., 2, 3, 4, 5, 6, 7, 8, 9, or 10 layers) of cultured cells which may or may not be equidistant. Individual layers or the layered formation as a whole may comprise a single cell type or multiple cell types. The layered formations as a whole may comprise a single cell type or multiple cell types (e.g., 2, 3, 2-3, 2-4 cell types). The cell types of individual layers and/or the multi-layered product can be any one of or a combination of myoblasts, myotubes, fibroblasts, endothelial cells, neurons, red blood cells, pre-adipocytes, induced pluripotent stem cells, and adipocytes.

Slurries used to produce layered products contain at least one texture agent that allows the slurry to achieve a semi-solid or solid form when set; they may-optionally further contain one or more of the functional and/or sensory agents as described above.

The non-fibrous layered products may be produced in a number of ways, but generally require the steps of forming the layers, which in turn requires dispensing then setting (solidifying) the corresponding slurry, Slurries used to produce layered products contain at least one texture agent that allows the slurry to achieve a semi-solid or solid form when set; they may-optionally further contain one or more of the functional and/or sensory agents as described above.

Layered products may be produced by separately forming and setting the individual layers as individual pieces and then assembling the pieces to create the layered product. In such cases, the layers can be allowed to passively adhere to each other, for example through electrostatic interactions such as hydrogen bonding. In the alternative, the layers may be bound together using binders or enzymatic methods, such as methods employing transglutaminases.

Layered products may also be produced as a single piece, by sequentially dispensing and setting layers of slurry to create a vertical stack. Layered products may also be produced by forming the layers simultaneously, for example as a slab, sheet, cylinder wherein the layers are immediately adjacent and aligned horizontally. Layered products having a marbled appearance may be produced by mixing or swirling two or more layers prior to setting them, or by dispensing successive slurry layers in manner that creates non-uniform, highly irregular layers. Combinations of these methods may also be used to create products having varied layering patterns.

Depending on the viscosity of the slurry used, the individual layers can be set sequentially or simultaneously.

Individual layers or layered pieces can be formed by dispensing the slurries into forms or onto a stationary or moving platform or conveyor using one or more stationary or movable dispensing devices to form discrete or continuous pieces. The layers or layered pieces may have the same or larger dimensions as the finished product, Where the pieces are larger than the dimensions of the final product, they may be processed into pieces having the desired final dimensions. Other methods are also possible.

In yet another embodiment, the layered products are produced using food manufacturing systems which deposit slurries sequentially one on top of another. In this method, the layers may be set simultaneously if high-viscosity slurries are used, or in cases where low-viscosity slurries are used, the layers are set sequentially following deposition of each layer and prior to deposition of the next layer. Slurries may be dispensed by a number of methods, as described above.

In yet another embodiment, individual layers are formed using pipe setting techniques that are known in the art.

### Methods of Manufacture

For the homogenous product, the cell slurry is mixed with above mentioned functional ingredients except texturizers for 5-15 min at 15-45 °C followed by the addition of texturizers at 45-75 °C and further mixing for 5-15 min. After final mixing the mixture is poured into molds and rapidly cooled down to form the homogenous block of the product. Finally, product is quick frozen to −80 °C and packaged into high barrier packaging. For the layered product with marbling as shown in the schematic, the marbling layer consisting of high fat emulsion and some texturizers to assist in forming product is prepared and kept warm until colored fraction is prepared. The colored mixture is prepared in the similar way to the homogenous product and then randomly layered with white marbling emulsion to get appearance as shown in the schematic.

Further details concerning the compositions, methods and systems herein described will become more apparent hereinafter from the following detailed invention of examples by way of illustration only, with reference to an experimental section.

### Examples

The cell-cultured edible food products, and related cells, compositions, and systems herein described are further illustrated in the following examples, which are provided by way of illustration and are not intended to be limiting.

### Example 1: Cell Slurry of In Vitro Cultured Fish Cells

In this example, cells derived from Bluefin tuna were concentrated into a viscous slurry that can be used as is, or frozen or dried, and stored prior to use. The cell slurry may contain one or more cell types, such as myoblasts, myotubes, pre-adipocytes, adipocytes, fibroblasts, endothelial cells, or induced pluripotent stem cells.

### Bluefin Tuna Cell Line Derivation

Pacific Bluefin tuna (12-100 pounds) were wild caught and identified visually and by genome sequencing. Myoblasts were harvested from muscle tissue. Typically, 6-24 grams of tissue were processed via enzyme and mechanical dissociation and seeded as 0.5-1.0 grams of tissue/well.

### Expansion of Bluefin Tuna Cells

Stable cell lines of Bluefin tuna cells were expanded under growth conditions. The cells proliferated in dilute conditions of approximately 10³ to 10⁶ cells per mL under growth pH and temperature conditions.

### Slurry Formation of Bluefin Tuna Cells

A concentrated cell slurry was formed through the concentration of cells from the dilute growth or differentiation solution. Concentrations were performed through centrifugation, settling, or other separation methods that separate the cells from the liquid. The slurry contained cell concentrations above 10⁶ per mL, and up to 109, depending on cell size. These slurries were viscous solutions akin to a suspension that may flow like a liquid. Optionally, the product was subsequently frozen or dried to form a solid product.

### Cell Slurry Products

Cell slurries are used in the manufacture of products that include but are not limited to nutritional supplements and food ingredients. A nutritional supplement is produced by filling capsules with freeze-dried cultured Bluefin tuna cells. Such capsules are used in the same manner as fish oil capsules but offer the advantage of having a uniform and reproducible lipid profile while being free of environmental contaminants. The capsules are made from animal-free materials such as cell-cultured gelatin, modified tapioca starch and/or vegetable cellulose such as hydroxypropylmethylcellulose (also known as hypromellose), the latter of which offers the advantage of delaying release of their contents until they reach the small intestine, thereby optimizing lipid absorption.

Cell slurries are used in fresh, frozen, or dried form as food ingredients, alone or in combination with other animal and/or plant materials, for the production of a variety of foods, such as baby foods, pet foods and other products.

### Example 2: Homogenous, Single Cell Type Products of In Vitro Cultured Bluefin Tuna Cells

Homogenous Bluefin tuna products are manufactured by combining Bluefin tuna cell slurries as described in Example 1 above with one or more food ingredients mentioned before. These homogenous products are formed as single pieces that are substantially uniform throughout the piece with respect to their sensory and functional attributes.

Single cell type products are manufactured using cell slurries of a single cell type, such as myoblasts, myotubes, pre-adipocytes, adipocytes, fibroblasts, endothelial cells, or induced pluripotent stem cells. Both the cell type and the form will depend on the intended purpose of the final product. In one example, homogeneous single cell type products are formed into blocks that are further processed into mince (see Example 4 below), or as sheets having uniform or variable thickness that are combined into layered products having the desired firmness, mouthfeel, nutrition, appearance or other product attributes (see Example 3 below).

### Texture Agents

Texture agents, and the concentration of each, are chosen to achieve a degree of firmness, cohesiveness, springiness and chewiness suitable for the product's intended purpose.

### Product Forms

Homogeneous in vitro cultured Bluefin tuna cell products may be formed in a wide variety of shapes, including but not limited to saku (blocks, i.e., uniform pieces suited for preparing sashimi and other sliced presentations, for pan searing or for further processing into mince, steaks, fillets and loins, sashimi, dice, slices and cubes.

### Functional Agents

In addition to firmness, certain texture agents further provide additional functional attributes including but not limited thermal stability and browning, which agents allow the piece to remain solid during heating and/or brown during cooking to create an appetizing appearance and/or flavor; in such cases the concentration of such agents are chosen to provide a product that is acceptably heat stable and/or that browns in a controlled manner, without being unpleasantly tough. Further, texture agents are in some cases selected in order to provide emulsion stabilization in addition to other sensory or functional attributes.

### Example 3: Homogenous, Multiple Cell Type Product of In Vitro Cultured Bluefin Tuna Cells

Homogenous multiple cell type products of in vitro cultured Bluefin tuna cells are produced using multiple cell type slurries from cultures containing one, two, three, four or more cell types. In the alternative, multiple cell type products are prepared from two, three, four or more single cell type slurries combined in proportions that are suited to the intended purpose of the final product.

Multiple cell type products comprising both myocytes and adipocytes are formulated to give a product having a fat content that mimics the fat content of wild-caught or farmed Bluefin tuna cuts characterized as otoro (high-fat), chutoro (medium-fat) or akami (lean).

### Example 4: Mince of In Vitro Cultured Bluefin Tuna Cells

Minces of in vitro cultured Bluefin tuna cells are optimally formed by processing large pieces formed from either single cell type or multiple cell type homogeneous products as described in Example 2 and Example 3 above.

Single cell type or multiple cell type slurries are combined with one or more texturizers or one or more binders the produce a piece having a texture optimized for processing by chopping into small pieces, the size of which will vary according to the intended use of the mince. In the alternative, the piece is partially or completely frozen as a preliminary step in order to facilitate chopping. Because chopping will result in “breaking” the gel formed by the texture agents, consequently releasing moisture, pieces intended for use in producing mince are formulated using small amounts of dry ingredients, such as sodium alginate, fiber, psyllium husk and the like to facilitate water retention.

### Example 5: Non-Fibrous, Layered, Single Cell Type Product of In Vitro Cultured Bluefin Tuna Cells

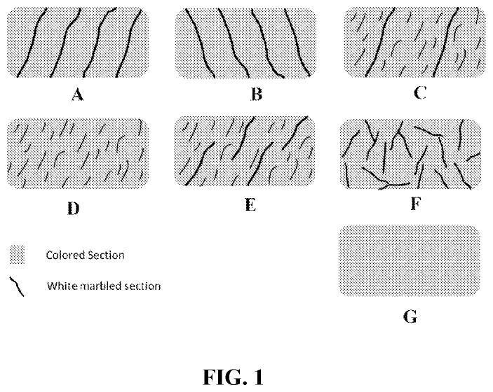

Non-fibrous, layered single cell type products of in vitro cultured Bluefin tuna cells are produced by first producing two or more individual layers of homogeneous single cell type products as described in Example 2 above and then combining the layers. See FIGS. 1A-1G.

The layered formation can be achieved in a number of ways, but in general require the steps of forming the layers, which in turn requires dispensing the corresponding slurry, then setting (solidifying) the layers, then assembling the multiple layers. Depending on the viscosity of the slurry used, the individual layers can be set sequentially or simultaneously, prior to or after assembling them. In one example, each layer is produced independently and allowed to set (i.e., solidify); the layers are then assembled by vertically stacking them. In this example, the layers can be formed by dispensing the slurries into forms or onto a platform as discrete or continuous pieces. Where the slurries are dispensed as continuous pieces onto a platform, the pieces can have the same length and width as the finished product, in which case they are set following dispensing, then assembled by vertical stacking. Where the pieces are larger than the dimensions of the final product, they can either be cut into smaller pieces, then set and stacked or in the alternative, the larger pieces can be set, then stacked to produce an intermediate form, and the intermediate form cut into multiple pieces having the desired length and width.

### Example 6: Non-Fibrous, Layered, Multiple Cell Type Products of In Vitro Culture Bluefin Tuna Cells

Non-fibrous, layered multiple cell type products of in vitro cultured Bluefin tuna cells are produced using individual layers that may each include one ore multiple cell types to produce a product that in the aggregate has two or more cell types. In addition to cell type composition, the layers contain one or more texture agents, which can be the same or different for each individual layer, and one or more additional functional and/or sensory agents as described in Example 2 above.

Marbling of the product can be achieved through the layering of this individual layers as mentioned earlier. The individual layers can be stabilized with food grade emulsifiers and can have individual formulation containing afore mentioned texturizing, coloring, and sensory ingredients.

### Example 7: Improved Refrigerated and Frozen Shelf Life of Product of In Vitro Cultured Bluefin Tuna Cells

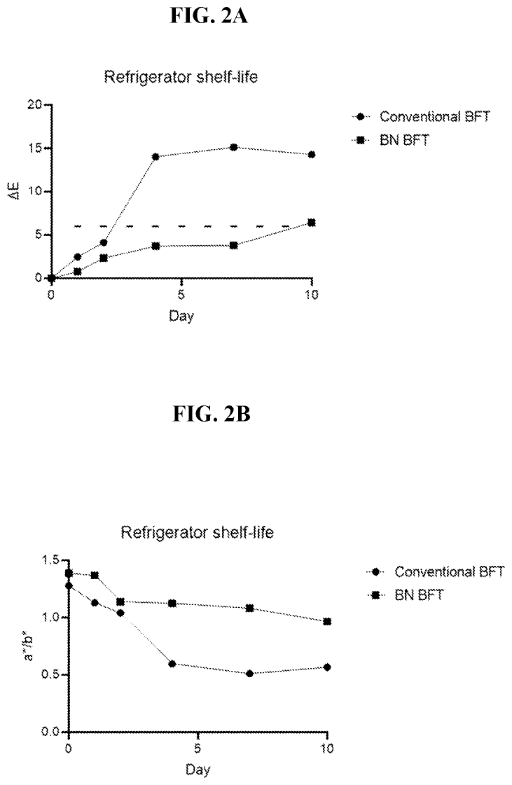

Non-fibrous layered product produced according to one or more of the above examples, was packed in parafilm and stored under refrigeration at 4 °C for 10 days. Thereafter, it was removed and reviewed for color degradation compared to conventional Bluefin tuna, which was used as a reference. As set forth in Ying Bu et al, “In general, the bright red color of tuna muscle is mainly associated with the presence of red pigments, especially oxymyoglobin (OxyMb). However, OxyMb converts to the brown MetMb during processing and storage . . . MetMb is mainly formed due to Mb oxidation, free radicals produced during lipid oxidation and trimethylamine oxide generation”. The overall color degradation of the tuna product according to the invention was at much slower rate with only 16% reduction in the a*/b* value. Referring to Ying Bu, this suggests a lower rate of lipid oxidation. FIGS. 2A and 2B show differences in color degradation under refrigerated conditions. FIG. 2A shows shelf-life as a function of ΔE value, while FIG. 2B shows the same data as a function of a*/b* value.

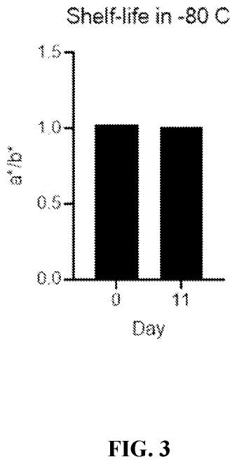

In another example, the product as used in above example, was placed in a freezer at −80 °C for 11 days then thawed for evaluation. The product packed in parafilm showed excellent freeze thaw stability with only <0.5% drip loss and retention of color attributes. The data shows there is no significant change in color of the product when stored in −80 °C for 11 days. FIG. 3 shows the shelf-life of cell-cultured Bluefin tuna product at −80 °C. This exemplifies that the cell cultured Bluefin tuna has a longer shelf-life compared to conventional Bluefin tuna.

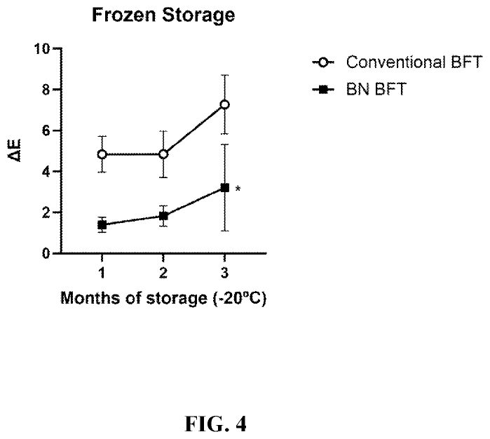

In another example, the product used as above was placed in a freezer at −20 °C for three months and compared to wild-caught Pacific Bluefin tuna (conventional Bluefin tuna). FIG. 4 shows differences in color degradation under −20 °C conditions. Color degradation as a function of ΔE value was significantly higher in conventional Bluefin tuna than cell-cultured Bluefin tuna samples indicating extended shelf-life of cell-cultured Bluefin tuna product under frozen storage conditions.

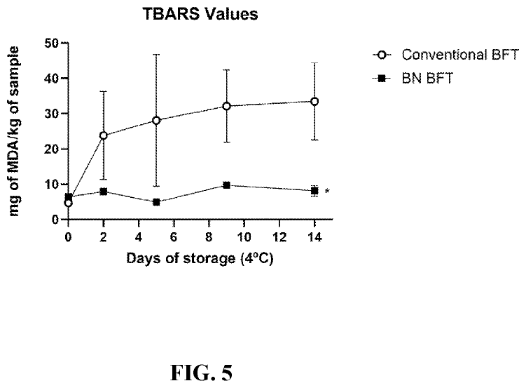

In another example, conventional Bluefin tuna and cell-cultured Bluefin tuna were vacuum packaged, stored under refrigeration at 4 °C for 14 days, and evaluated for lipid oxidation measured as a function of Malondialdehyde (MDA) production using a modified protocol described in Quevedo et al. (2013), Scheffler et al. (2010), and Sonar et al. (2020). FIG. 5 shows a significant decrease in the amount of MDA produced in cell-cultured Bluefin tuna compared to conventional Bluefin tuna indicating enhanced shelf-life retention for the cell-cultured Bluefin tuna product. Both samples were also evaluated for heavy metals including arsenic, cadmium, lead, and mercury, by ICP-MS (Table 2). Cell-cultured Bluefin tuna had significantly lower contaminant levels compared with wild-caught and ranched conventional Bluefin tuna.

The examples set forth above are provided to give those of ordinary skill in the art a complete invention and description of how to make and use the embodiments of edible compositions comprising Bluefin tuna cells that are substantially free of contaminants, and related compositions, methods, and systems of the invention, and are not intended to limit the scope of what the inventors regard as their invention. Those skilled in the art will recognize how to adapt the features of the exemplified cells, compositions, methods and systems herein disclosed to additional cells, compositions, methods and systems according to various embodiments and scope of the claims. All patents and publications mentioned in the specification are indicative of the levels of skill of those skilled in the art to which the invention pertains.

The entire disclosure of each document cited (including patents, patent applications, journal articles, abstracts, laboratory manuals, books, or other inventions) in the Background, Summary, Detailed Description, and Examples is hereby incorporated herein by reference. All references cited in this invention are incorporated by reference to the same extent as if each reference had been incorporated by reference in its entirety individually. However, if any inconsistency arises between a cited reference and the present invention, the present invention takes precedence.

The terms and expressions, which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the invention has been specifically disclosed by embodiments, exemplary embodiments and optional features, modification and variation of the concepts herein disclosed may be resorted to by those skilled in the art, and that such modifications and variations are considered to be within the scope of this invention as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the invention pertains.

When a Markush group or other grouping is used herein, all individual members of the group and all combinations and possible sub-combinations of the group are intended to be individually included in the invention. Every combination of components or materials described or exemplified herein can be used to practice the invention, unless otherwise stated. One of ordinary skill in the art will appreciate that methods, system elements, and materials other than those specifically exemplified may be employed in the practice of the invention without resort to undue experimentation. All art-known functional equivalents, of any such methods, device elements, and materials are intended to be included in this invention. Whenever a range is given in the specification, for example, a temperature range, a frequency range, a time range, or a composition range, all intermediate ranges and all subranges, as well as, all individual values included in the ranges given are intended to be included in the invention. Any one or more individual members of a range or group disclosed herein may be excluded from a claim of this invention. The invention illustratively described herein suitably may be practiced in the absence of any element or elements, limitation or limitations which is not specifically disclosed herein.

A number of embodiments of the invention have been described. The specific embodiments provided herein are examples of useful embodiments of the invention and it will be apparent to one skilled in the art that the invention can be carried out using a large number of variations of the genetic circuits, genetic molecular components, and methods steps set forth in the present description. As will be obvious to one of skill in the art, methods and systems useful for the present methods and systems may include a large number of optional composition and processing elements and steps.

In particular, it will be understood that various modifications may be made without departing from the spirit and scope of the present invention. Accordingly, other embodiments are within the scope of the following claims.

### REFERENCES

Abnet, 2007Abnet C. (2007). Carcinogenic food contaminants. Cancer Invest. 25, 189-196. 10.1080/07357900701208733
Androutsopoulos et al., 2013 Androutsopoulos V., Hernandez A., Liesivuori J., Tsatsakis A. (2013). A mechanistic overview of health associated effects of low levels of organochlorine and organophosphorous pesticides. Toxicology 307, 89-94. 10.1016/j.tox.2012.09.011
Bassil K., Vakil C., Sanborn M., Cole D., Kaur J., Kerr K. (2007). Cancer health effects of pesticides. Can. Fam. Phys. 53, 1704-1711
Chiesa L M, Labella G F, Panseri S, Pavlovic R, Bonacci S, Arioli F. Distribution of persistent organic pollutants (POPS) IN wild Bluefin tuna (Thunnus thynnus) from different FAO capture zones. Chemosphere. 2016 June; 153:162-9. doi: 10.1016/j.chemosphere.2016.03.010. Epub 2016 Mar 24. PMID: 27016811.
Claudia Ruiz-Capillasnd and Ana M. Herrero, (2019). Impact of Biogenic Amines on Food Quality and Safety. Foods 8(2) 62.
Grandjean P., Landrigan P. (2006). Developmental neurotoxicity of industrial chemicals. Lancet 368, 2167-2178. 10.1016/S0140-6736(06)69665-7
Grunwald, N. Tatiyaborworntham, C. Faustman, M. P. Richards, “Effect of 4-hydroxy-2-nonenal on myoglobin-mediated lipid oxidation when varying histidine content and hemin affinity”, Food Chemistry, 227 (2017), pp. 289-297, 10.1016/j.foodchem.2017.01.035
Wang, J.; Zhang, H.; Xie, J.; Yu, W.; Sun, Y. (2022). Effects of Frozen Storage Temperature on Water-Holding Capacity and Physicochemical Properties of Muscles in Different Parts of Bluefin Tuna. Foods 2022, 11, 2315. https://doi.org/10.3390/foods11152315
Kher S., De Jonge J., Wentholt M., Deliza R., de Andrade J., Cnossen H., et al. (2011). Consumer perceptions of risks of chemical and microbiological contaminants associated with food chains: a cross-national study. Int. J. Consum. Stud. 37, 73-83. 10.1111/j.1470-6431.2011.01054.x
Khan et al., 2008; Khan S., Cao Q., Zheng Y., Huang Y., Zhu Y. (2008). Health risks of heavy metals in contaminated soils and food crops irrigated with wastewater in Beijing, China. Environ. Pollut. 152, 686-692. 10.1016/j.envpol.2007.06.056
Schantz et al., 2004 Schantz S., Gardiner J., Gasior D., McCaffrey R., Sweeney A., Humphrey H. (2004). Much ado about something: the weight of evidence for PCB effects on neuropsychological function. Psychol. Schools 41, 669-679. 10.1002/pits.20008
Shelf Life of Foods: Guidelines for Its Determination and Prediction, 1993;
Singh, Benjakul, Zhou, Zhang, & Deng, 2021—Effect of squid pen chitooligosaccharide and epigallocatechin gallate on discoloration and shelf-life of yellowfin tuna slices during refrigerated storage. Food Chemistry, 351 (10) (2021), p. 129296, 10.1016/j.foodchem.2021.129296
Tseng et. al., 2021 Bluefin tuna reveal global patterns of mercury pollution and bioavailability in the world's oceans, Proceedings of the National Academy of Sciences, September 2021 DOI: 10.1073/pnas.2111205118;
Ying Bu et al., 2022; Changes in quality characteristics of southern bluefin tuna (Thunnus maccoyii) during refrigerated storage and their correlation with color stability, LWT, Volume 154, 15 Jan. 2022, 112715

1. An edible composition comprising a homogenous mixture of in vitro cultured fish cells, wherein said cells are each less than from 20 μm to 200 μm in diameter and are substantially free of one or more contaminants.

2. The edible composition according to claim 1, wherein said cells are each less than 20 μm in diameter and are substantially free of one or more contaminants.

3. The edible composition according to claim 1, wherein said cells are each less than 200 μm in diameter and are substantially free of one or more contaminants.

4. The edible composition according to claim 1, wherein said contaminants are environmental contaminants.

5. The edible composition according to claim 4, wherein said environmental contaminants are selected from the group consisting of mercury, lead, cadmium, zinc, copper, nickel, chromium, arsenic, aluminum, fluoride, radon, persistent organic pollutants and pesticides.

6. The edible composition according to claim 1, wherein said composition comprises one, two, or three cell types.

7. The edible composition of claim 6, wherein said cell type is selected from the group consisting of myoblasts, myotubes, fibroblasts, endothelial cells, neurons, red blood cells, pre-adipocytes, induced pluripotent stem cells, and adipocytes.

8. The edible composition according to claim 1, wherein said composition is in the form of a viscous slurry or mince.

9. The edible composition according to claim 1, wherein said composition is frozen or freeze-dried.

10. The edible composition according to claim 1, wherein said composition is a liquid, semi-liquid, semi-solid, solid, or foam.

11. The edible composition according to claim 1, wherein said composition contains less than 0.1 parts per million [ppm] of the one or more contaminants.

12. The edible composition of claim 11, wherein said contaminant is mercury.

12. An edible composition comprising a collection of non-fibrous in vitro cultured fish cells, wherein said cells are in a layered formation and substantially free of contaminants, and each layer comprises a homogenous mixture of cells.

13. The edible composition according to claim 1, wherein said composition comprises one cell type.

14. The edible composition according to claim 1, wherein said composition comprises between 1-3 cell types.

15. An edible composition comprising a homogenous mixture of in vitro cultured fish cells derived from the genus Thunnus, wherein said cells are each less than from 20 μm to 200 μm in diameter and are substantially free of one or more contaminants.

16. An edible composition comprising a homogenous mixture of in vitro cultured fish cells derived from Bluefin tuna, wherein said cells are each less than from about 20 μm to about 200 μm in diameter and are substantially free of one or more contaminants.

17. An edible composition comprising a homogenous mixture of in vitro cultured fish cells derived from Bluefin tuna, wherein said cells are each less than 20 μm in diameter and are substantially free of one or more contaminants.

18. The edible composition according to claim 16, wherein said tuna comprises a Toro cut.

19. The edible composition according to claim 16, wherein said tuna comprises an akami cut.

20. The edible composition according to claim 16, comprising a color profile with L 46.56-70.59, a* 12.21-38.76 and b* 1.24-61.68.

21. The edible composition according to claim 16, comprising a concentration of color from 0.001% to 5% to achieve L*, a*, and b* values in the range of 33.0-62.8, 4.77-21.9, and 4.72-15.4, respectively.

22. The edible composition according to claim 16 wherein said edible composition comprises a refrigerated shelf life of 4-14 days.

23. The edible composition according to claim 16, wherein said edible composition comprises a frozen shelf life of 90-120 days.

24. The edible composition according to claim 16, wherein said edible composition is a single cell consistent product.

25. The edible composition according to claim 24, wherein said single cell consistent product comprises a single cell type or a multiple cell type.

26. The edible composition according to claim 24, wherein said product is marbled.

27. An edible composition comprising a collection of non-fibrous in vitro cultured fish cells derived from Bluefin tuna, wherein said cells are in a layered formation and substantially free of contaminants, and each layer comprises a homogenous mixture of cells.

28. The edible composition according to claim 16, wherein said edible composition comprises a longer shelf life than conventional Bluefin tuna.

29. The edible composition according to claim 23, wherein said edible composition comprises a longer shelf life than conventional Bluefin tuna.
